SEQUENCE LISTING (6 sequences)

SEQ ID NO: 1 - DNA, 22 bp, Artificial Sequence
  Description of Artificial Sequencehepatocyte growth factor (HGF) PCR sense primer
ctgatccaaacatccgagttgg

SEQ ID NO: 2 - DNA, 24 bp, Artificial Sequence
  Description of Artificial Sequencehepatocyte growth factor (HGF) PCR antisense primer
aggtgtggtatcaccttcacaacg

SEQ ID NO: 3 - DNA, 20 bp, Artificial Sequence
  Description of Artificial Sequencec-met proto-oncogene (HGF receptor) PCR sense primer
tcctcgtgctcctgtttacc

SEQ ID NO: 4 - DNA, 22 bp, Artificial Sequence
  Description of Artificial Sequencec-met proto-oncogene (HGF receptor) PCR antisense primer
tctttcgtttcctttagccttc

SEQ ID NO: 5 - DNA, 20 bp, Artificial Sequence
  Description of Artificial Sequencebeta-actin internal control PCR sense primer
aagagaggcatcctcaccct

SEQ ID NO: 6 - DNA, 20 bp, Artificial Sequence
  Description of Artificial Sequencebeta-actin internal control PCR antisense primer
tacatggctggggtgttgaa

(19) United States
(12) Patent Application Publication
(10) Pub. No.: US 2005/0026830 A1
(43) Pub. Date: Feb. 3, 2005

(54) Compositions and methods for treating fibrosis

(76) Inventor: Yasushi Kawaguchi, Shinjuku-ku (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/846,223

(22) Filed: May 13, 2004

Related U.S. Application Data
(60) Provisional application No. 60/470,685, filed on May 14, 2003.

(51) Int. Cl. A61K 38/18

(52) U.S. Cl. 514/12

(57) ABSTRACT

The present invention provides medicaments, and methods for their use, that produce a non-physiologically high level of HGF at the site of a fibrosis plaque. The high level of HGF is unexpectedly found to inhibit procollagen production by abnormal fibroblasts responsible for formation of the fibrosis plaque. The present invention also provides methods for identifying individuals susceptible to fibrosis.

### CROSS-REFERENCES TO RELATED APPLICATIONS

This application claims the benefit of U.S. Provisional Application No.60/470,685 filed May 14, 2003, which is incorporated herein by reference.

### FIELD OF THE INVENTION

The present invention relates to the field of biochemistry, and gene therapy, particularly to methods for preventing and treating fibrosis. The invention provides medicaments, and methods for their use, that produce a non-physiologically high level of HGF at the site of a fibrosis plaque. The high level of HGF is unexpectedly found to inhibit procollagen production by abnormal fibroblasts that over-produce fibrous tissue. The present invention also provides methods for identifying individuals susceptible to fibrosis.

### BACKGROUND OF THE INVENTION

Hepatocyte growth factor (HGF) was first described as a potent mitogen for adult rat hepatocytes in primary culture. Nakamura T, et al., Proc Natl Acad Sci USA 83:6389-93 (1986). HGF has also been described as a potent mitogen for various types of epithelial cells (Matsumoto K, Nakamura T., J. Biochem 1996;119: 591-600). Moreover, HGF is synthesized and secreted by mesenchymal cells such as macrophages (Camussi G, et al., J Immunol 1997;158: 1302-9), endothelial cells (Noji S, et al., Biochem Biophys Res Commun 1990; 173: 42-7), and fibroblasts (Gohda E, et al., Cytokine 1994; 6: 633-40). Considerable evidence has also accumulated indicating that HGF has important functions in vivo as a hepatotrophic factor during the regenerative events in liver injured by partial hepatectomy or by hepatotoxin treatment. Ishiki Y, et al., Hepatology 1992; 16: 1227-35. HGF may, therefore, play a critical role in triggering or modulating proliferation of remaining hepatocytes.

Consistent with the pharmacological effects of HGF, the cellular receptor for HGF has been identified as the c-met protein, a transmembrane tyrosine kinase receptor that transmits an array of important cellular responses induced by HGF (Bottaro D P, et al., Science 1991;251:802-4). The c-met was originally described as an activated oncogene expressed on a human osteosarcoma cell line (Testa J R, et al., Oncogene 1990;5:1565-71).

In addition to synthesizing and secreting HGF, a wide variety of mesenchymal cells, including fibroblasts(Li D, Tseng S C G, J Cell Physiol 1997;171:361-72), renal epithelial cells (Igawa T, et al., Biochem Biophys Res Commun 1991;174:831-8), and synovial cells (Koch A E, et al, Arthritis Rheum 1996;39: 1566-75), have been revealed to be HGF targets, having multiple effects in regulating cell proliferation, migration, morphogenesis, development and regeneration (Vande Woude G F., Jpn J Cancer Res 1992;83:227-32). HGF is also reported to protect against liver cirrhosis (Matsuda Y, et al., Hepatology 1997;26:81-9), pulmonary fibrosis (Yaekashi M, et al., Am J Respir Clin Care Med 1997; 156: 1937-44), and glomerulosclerosis (Mizuno S, et al., J Clin Invest 1998;101:1827-34) in vivo.

Despite its anti-fibrotic properties, serum levels of HGF are markedly increased in several fibrotic disorders including SSc (Kawaguchi Y., et al., J Rheumatol 1999;26:1012-3), hepatic failure (Tsubouchi H, et al., Lancet 1992;340:307), pulmonary fibrosis (Hojo S, et al., Respiratory Med 1997;91:511-6), and renal fibrosis (Takada S, et al. Transplant Int 1996; 9: 151-4). In previous studies, increased levels of serum HGF were observed in SSc patients (Kawaguchi Y, et al., J Rheumatol 1999;26:1012-3) as well as several fibrotic disorders including fulminant hepatitis (Tsubouchi H, et al., Lancet 1992;340:307), pulmonary fibrosis (Hojo S, et al., Respiratory Med 1997;91:511-6), and renal injuries (Takada S, et al., Transplant Int 1996; 9: 151-4).

### SUMMARY OF THE INVENTION

Generally, mesenchymal cells such as fibroblasts produce HGF but do not express c-met protein spontaneously (Kajihara T, et al., Arch Oral Biol 1999;44: 135-47). However, studies leading to the present invention show that SSc fibroblasts produce both HGF and c-met protein. Further, the present study reveals fibroblasts in fibrotic tissue of SSc patients exhibit an ability to increase HGF production. These observations suggest that increased levels of serum HGF in patients with SSc results from excessive production of HGF by SSc fibroblasts.

As SSc fibroblasts over-express HGF leading to abnormally high serum levels, it was unexpected to find that treating fibroblasts locally with even higher, non-physiological concentrations of HGF actually inhibited procollagen deposition (FIG. 6) and formation of a collagen mass characteristic of fibrosis disease. The present invention exploits these observations to provide medicaments and methods for identifying, preventing and treating fibrosis.

Accordingly, one embodiment of the invention provides methods of treating or preventing fibrosis that include administering a medicament to a mammal, including fibrosis associated with diseases such as systemic sclerosis, scleroderma, dermatosclerosis, sclerosis corii, sclerosis cutanea, localized scleroderma, morphea, scleroderma circumscriptum, sclerodermatitis, hidebound disease, and skin bound disease. Preferably the medicament is administered prior to damage to a tissue near a forming collagen mass characteristic of fibrosis, more preferably prior to the formation of a collagen mass characteristic of fibrosis.

The medicament includes HGF in an amount effective to inhibit collagen formation and a pharmaceutically acceptable excipient. The amount of HGF effective in inhibiting collagen formation is between about 0.001 mg and about 50 mg per patient, preferably about 0.01 mg and about 10 mg per day per patient, and more preferably about 0.05 mg and about 5 mg per day per patient. The medicament may further include other pharmaceutically active ingredients, such as anti-inflammatory agent(s). In some aspects of this embodiment mimetics or fusion proteins of HGF are used as these variants increase the half-life of HGF in vivo after administering the medicament to a mammal.

The medicament may be administered by direct application, systemic injection, nebulized inhalation, or oral ingestion, and may be administered in a single dose, or multiple doses over a period of time. Administration of the medicament is preferably to a mammal, more preferably a human.

Other embodiments of the invention are medicaments that include a vector including a nucleic acid comprising a nucleotide sequence encoding HGF. Introducing these medicaments to a subject results in expression and secretion of HGF protein in an amount effective to inhibit collagen formation. In addition to the vector, these medicaments also include a pharmaceutically acceptable excipient, and optionally contain other pharmaceuticals, such as anti-inflammatory agent(s). In one aspect, the vector is an EJV virus.

The invention also includes methods for treating or preventing fibrosis using the vector medicaments described above. These methods can include administering the medicament, which includes a technique selected from the group consisting of direct application, systemic injection, nebulized inhalation, and oral ingestion, and are suitable for treating the same ranges of diseases noted above. As above, administration of the vector medicaments may be in single doses or through multiple doses over a period of time. Preferably dosing is initiated prior to damage to a tissue near a forming collagen mass characteristic of fibrosis, more preferably prior to the formation of a collagen mass characteristic of fibrosis.

Further embodiments include cell-based medicaments. These embodiments have a mesenchymal preparation of one or more cells each cell having a nucleic acid including a nucleotide sequence encoding HGF, and a pharmaceutically acceptable excipient. The mesenchymal preparation of these embodiments may be pluripotent stem cells or fibroblasts. The medicaments may also include other pharmaceutically active ingredients, as described above.

Introduction of these medicaments to a mammal results in expression and secretion of HGF by the cells of the mesenchymal preparation in an amount effective to inhibit collagen formation. This amount of HGF is between about 0.001 mg and about 50 mg per day per patient, preferably about 0.01 mg and about 10 mg per day per patient, and more preferably about 0.05 mg and about 5 mg per day per patient.

Methods for using the cell-based medicaments are also provided. These methods include administering the medicament to a mammal through one dose or multiple doses over a period of time. Administering the medicament may be done by any of the techniques provided herein, and in the time-frame previously described, but is preferably done prior to manifestation of fibrosis and by contacting the tissue(s) having fibrosis directly with the medicament.

Still other embodiments provided by the invention are methods for early diagnosis of fibrosis. These methods involve detecting the expression of HGF receptor in fibroblasts isolated from a mammal, such as the c-met protooncogene. Not observing expression of HGF receptor indicates that the mammal does not have fibrosis. In contrast, detecting the c-met protein is indicative of a mammal susceptible to fibrosis. The preferred method for detection of HGF receptor transcript is detecting the corresponding mRNA using PCR detection using total RNA isolated from fibroblasts taken from a mammal susceptible to, or symptomatic for, fibrosis.

The invention also provides articles of manufacture, for example, an article having a medicament with HGF as the active ingredient and instructions as to administration of the medicament in a manner and amount sufficient to inhibit formation of a collagen mass characteristic of fibrosis. The amount sufficient to obtain this result is as noted above. These articles may optionally include an inhaler.

Still other articles of manufacture have a medicament that includes a vector having a nucleic acid including a nucleotide sequence encoding HGF, and instructions as to administration of the medicament in a manner and amount sufficient to inhibit formation of a collagen mass characteristic of fibrosis.

Another article of manufacture includes a medicament with a mesenchymal preparation having one or more cells each having a nucleic acid comprising a nucleotide sequence encoding HGF, and instructions as to culturing and administering the medicament in a manner and amount sufficient to inhibit collagen formation.

### BRIEF DESCRIPTION OF THE DRAWINGS

FIG. 1 is an analysis of mRNA expression of HGF and c-met in skin fibroblasts. Lane M is a DNA marker ladder (100 bp).

FIG. 2 is a graphical depiction of HGF production of SSc (white) and normal (black) fibroblasts.

FIG. 3 is a graphical depiction of the effect of exogenous IL-1α on HGF production by fibroblasts taken from patients with (white) and without (black) SSc.

FIG. 4 is a micrograph showing localization of c-met protein in fibroblast monolayers using immunostaining.

FIG. 5 is an electrophoretic analysis of mRNA expression of c-met in skin fibroblasts transfected with the IL-1α gene.

FIG. 6 graphically illustrates procollagen type I production by skin fibroblasts stimulated with HGF

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Collagen formation” refers to the synthesis of a protein substance as exemplified by the white fibers (collagenous fibers) of skin, tendon, bone, cartilage and all other connective tissue. Collagen is composed of molecules of tropocollagen, which is formed from procollagen. “Procollagen” is a triple helical trimer of collagen molecules having terminal extension peptides that are linked by disulphide bridges. The terminal peptides are later removed by specific proteases to produce a tropocollagen molecule.

In disease states, and in some instances of physical or chemical injury, excessive collagen may be formed in the affected tissue. This excessive build up of collagen, or collagen mass, is characteristic of fibrosis. In many tissues, including the eye, lungs, vascular tissue, and joints, the collagen mass can cause damage to the surrounding tissue that occasionally proves debilitative.

“Fibrosis” refers to the formation of excessive fibrous tissue, as in a reparative or reactive process. One of the principle fibrous tissues formed in excess during the course of fibrosis is collagen. Fibrosis can occur in response to physical or chemical injury to a tissue, or can be the result of abnormal tissue response and/or physiology, such as occurs in some disease states. Examples of disease states where fibrosis occurs include systemic sclerosis, scleroderma, dermatosclerosis, sclerosis corii, sclerosis cutanea, localized scleroderma, morphea, scleroderma circumscriptum, sclerodermatitis, hidebound disease and skin bound disease.

“HGF” is a physiologically active 83.1 kDa peptide having 728 amino acids and exhibiting diverse pharmacological activities (See, e.g., GenBank Acc. No: NM_{—}000601). The gene encoding HGF has been sequenced and the protein's pharmacological activities identified, as described in, e.g., JIKKEN-IGAKU (Experimental Medicine), Vol. 10, No. 3 (extra issue), 330-339 (1992); Nakamura T., et al., Nature 342:440-443(1989); and Cioce V, et al., J Biol Chem 1996 May 31;271(22):13110-5. In view of its pharmacological activities, HGF may be useful in disease treatments as described, for example, in Japanese Patent KOKAI (Laid-Open) Nos. 4-18028 and 4-49246, EP 492614, Japanese Patent KOKAI (Laid-Open) No. 6-25010, WO 93/8821, Japanese Patent KOKAI (Laid-Open) Nos. 6-172207, 7-89869 and 6-40934, WO 94/2165, Japanese Patent KOKAI (Laid-Open) Nos. 6-40935, 6-56692 and 7-41429, WO 93/3061, and Japanese Patent KOKAI (Laid-Open) No. 5-213721.

HGF of the present invention also includes “fuision proteins”, e.g., covalent attachment of the constant regions of immunoglobulins, or other proteins or peptides resistant to degradation in vivo. By combining HGF with a degradation-resistant protein, the half-life of HGF activity in vivo is increased.

The “HGF receptor” has been identified as the product of the c-Met proto-oncogene [Bottaro et al., Science, 251:802-804 (1991); Naldini et al., Oncogene, 6:501-504 (1991); WO 92/13097 published Aug. 6, 1992; WO 93/15754 published Aug. 19, 1993]. The receptor is usually referred to as “c-Met” or “p190^{MET}” and typically comprises, in its native form, a 190-kDa heterodimeric (a disulfide-linked 50-kDa α-chain and a 145-kDa β-chain) membrane-spanning tyrosine kinase protein [Park et al., Proc. Natl. Acad. Sci. USA, 84:6379-6383 (1987)]. Several truncated forms of the c-Met receptor have also been described [WO 92/20792; Prat et al., Mol. Cell. Biol., 11:5954-5962 (1991)].

“Anti-inflammatory agent” refers to any compound or combination of compounds intended to reduce inflammation. Exemplary anti-inflammatory agents include salicylates, corticosteroids, NANSAIDS such as rofecoxib, naproxen, ibuprofen and other nonsteroidal anti-inflammatory drugs as discussed in Ray, et al., The Lancet 359, pp. 118-123 (2002).

“Half-life” refers to the time required for half the quantity of a drug or other substance deposited in a living organism to be metabolized or eliminated by normal biological processes.

Medicaments of the present invention, and compositions containing these compounds, may be applied to a tissue affected by fibrosis in a variety of ways. As used herein, “direct application” refers to contacting the compound directly to affected tissue. “Systemic injection” refers to injection at a site distant from the wound site to be treated. Systemic injection includes intravenous, subcutaneous and intramuscular injection. “Nebulized inhalation” refers to dispersing a liquefied medicament of the invention in fine droplets, which are then inhaled. Nebulized inhalation is particularly useful for treatment of pulmonary tissue, or the medicament can be absorbed into the bloodstream and transported to a distant site of fibrosis via the vascular system. Transdermal refers to application of medicaments of the invention directly to the skin, e.g., through application of an ointment, creme or in time-release forms that can be rubbed or placed on the skin surface.

“Vector” refers to any type of genetic construct containing a nucleic acid capable of self-replication or being transcribed in a cell. Vectors used for the amplification of nucleotide sequences (both coding and non-coding) are also encompassed by the definition. In addition to the coding sequence, vectors will generally include restriction enzyme cleavage sites and the other initial, terminal and intermediate DNA sequences that are usually employed in vectors to facilitate their construction and use. The expression vector can be part of a plasmid, virus, or nucleic acid fragment.

“Mesenchymal preparation” refers to any pharmaceutically acceptable substance that includes mesenchymal cells. Mesenchymal cells are fusiform or stellate cells found between the ectoderm and endoderm of young embryos. Most mesenchymal cells are derived from established mesodermal layers, but in the cephalic region they also develop from neural crest or neural tube ectoderm. Mesenchymal cells are pluripotential cells in the embryonic body, developing at different locations into any of the types of connective or supporting tissues, to smooth muscle, to vascular endothelium, and to blood cells.

Mesenchymal cells include “stem cells,” which are unspecialized cells that renew themselves for long periods through cell division. Under certain physiologic or experimental conditions, stem cells can be induced to become specialized cells, such as the beating cells of the heart muscle or the insulin-producing cells of the pancreas.

“Fibroblasts” are a type of mesenchymal cell that differentiate to form chondroblasts, collagenoblasts, and osteoblasts.

“Transduced” refers to the transfer of foreign nucleic acid into a cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes ofthis invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The terms “peptide” and “protein” are used herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Peptides and proteins of the present invention include amino acid polymers having D- and L-isoforms of individual amino acid residues, as well as other amino acid variants, as described herein. Peptides are distinguished by the number of amino acid residues making up the primary structure of the molecule. For purposes of this invention, peptides are those molecules comprising up to 50 amino acid residues, and proteins comprise 50 or more amino acid residues. However, methods of synthesis and/or delivery of peptides and proteins of the invention are similar, if not identical, as will be appreciated by one of skill in the art. Therefore, where appropriate, these terms are synonymous when discussing methods of synthesis, modification or use as therapeutic or diagnostic reagents.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, o-phosphoserine, and phosphothreonine, in addition to amidated and sulphonated amino acids. “Amino acid analog” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. In the context of the present invention, inclusion of amino acid mimetic in the amino acid sequence of HGF increases the half-life of the HGF protein in vivo, and in some circumstances enhances HGF activity.

Amino acids may be referred to herein by either commonly known three letter symbols or by one-letter symbols recommended by the IUPAC-IUB Biochemical Nomenclature Commission. Nucleotides, likewise, may be referred to by their commonly accepted single-letter codes.

“Amino acid sequence” refers to the positional relationship of amino acid residues as they exist in a given polypeptide or protein.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-o-methyl ribonucleotides and peptide-nucleic acids (PNAs).

Unless otherwise indicated, a particular nucleic acid sequence also implicitly encompasses conservatively modified variants thereof (e.g., degenerate codon substitutions, see below) and complementary sequences, as well as the sequence explicitly indicated.

“Conservatively modified variants” refers to those nucleic acids that encode identical or essentially identical amino acid sequences, or where the nucleic acid does not encode an amino acid sequence, to essentially identical sequences. The term also refers to fragments of particular sequences, where the sequence of the fragment has been conservatively modified as described herein. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and UGG, which is ordinarily the only codon for tryptophan) can be modified to yield a functionally identical molecule. Accordingly, each silent variation of a nucleic acid that encodes a polypeptide is implicit in each described sequence. (See e.g., Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “coding sequence”, in relation to nucleic acid sequences, refers to a plurality of contiguous sets of three nucleotides, termed codons, each codon corresponding to an amino acid as translated by biochemical factors according to the universal genetic code, the entire sequence coding for an expressed protein, or an antisense strand that inhibits expression of a protein. A “genetic coding sequence” is a coding sequence where the contiguous codons are intermittently interrupted by non-coding intervening sequences, or “introns.” During mRNA processing intron sequences are removed, restoring the contiguous codon sequence encoding the protein or anti-sense strand.

“Total RNA” refers to the entire spectrum of RNA molecules that can be isolated from a cellular system.

“Excipient” refers to an inert substance used as a diluent or vehicle for a drug.

### DETAILED DESCRIPTION

### I. Introduction

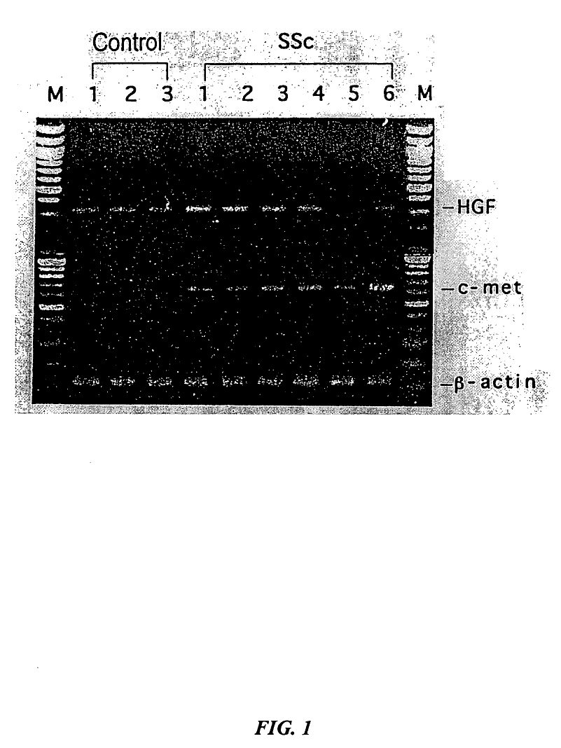

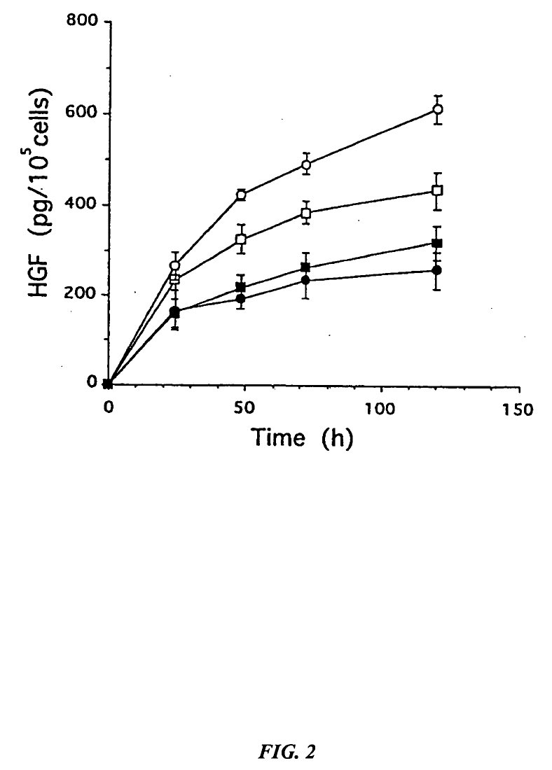

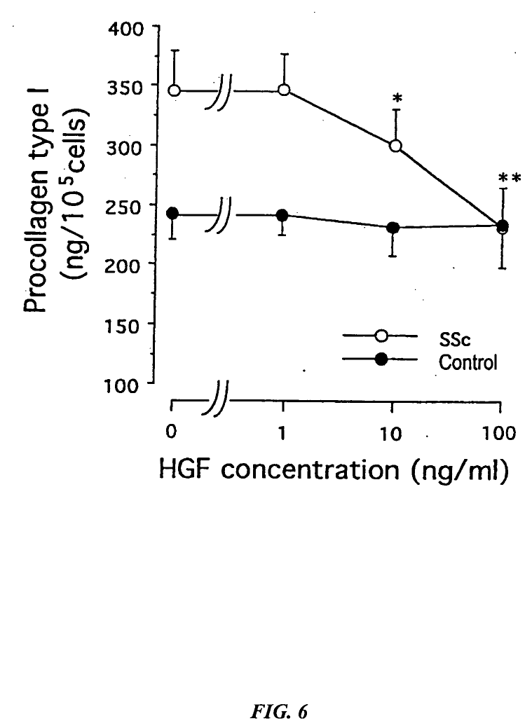

The present invention provides medicaments and methods for preventing and treating fibrosis in, for instance, diseases such as systemic sclerosis, scleroderma, dermatosclerosis, sclerosis corii, sclerosis cutanea, localized scleroderma, morphea, scleroderma circumscriptum, sclerodermatitis, hidebound disease and skin bound disease. These medicaments and methods are based on the finding that HGF receptor, c-met, is associated with SSc fibroblasts known to deposit excess fibrous tissue, particularly collagen, forming collagen plaques characteristic of fibrosis (see, e.g., FIG. 1). The same SSc fibroblasts expressing c-met also produce elevated amounts of HGF (FIG. 2), leading to elevated HGF serum levels in patients suffering from fibrosis. It was therefore an unexpected result to find that high concentrations of HGF inhibit procollagen production by SSc fibroblasts (FIG. 6).

The medicaments of the present invention are designed to deliver suitably high HGF levels to the site of fibrosis and inhibit procollagen production by SSc fibroblasts. Preferably the medicament is administered prior to damage to a tissue near a forming collagen mass characteristic of fibrosis, more preferably prior to the formation of a collagen mass characteristic of fibrosis.

In addition to allowing for therapeutic levels of HGF at a site of fibrosis, the methods and medicaments of the present invention may also be manipulated to deliver HGF at a designated time and for a determinable duration. For example, some embodiments are protein-based with a finite half-life in vivo. Other embodiments provide pluripotent cells that can be induced to reside in a diseased tissue, thereby preventing the formation of collagen masses characteristic of fibrosis. These embodiments and others are described in more detail below.

In addition to being effective therapeutics in treatment of fibrosis, the medicaments of the present invention also have utility in preventing the initial formation the collagen mass characteristic of fibrosis. In this way, the medicaments of the present invention may provide a means for at least rendering fibrosis asymptomatic for an extended period, if not indefinitely, and slowing the progress of a pre-existing condition.

To provide a more effective preventative course, practitioners are preferably provided with methods for identifying patients that are susceptible to fibrosis prior to symptomatic display of the condition. Accordingly, the present invention provides methods for the identification of individuals susceptible to fibrosis, and for early detection of fibrosis in suspect tissues prior to the formation of a collagen mass characteristic of fibrosis. This aids in minimizing the impact of the disease and limiting, if not abolishing, damage to tissue near a forming collagen mass, as occurs in some organs through the course of the disease.

### II. Sources of Hepatocyte Growth Factor

The following sections provide sources of HGF or nucleic acids encoding HGF that can be used as ingredients in forming the medicaments of the invention.

A. Proteins

Some embodiments of the present invention use HGF protein as the active ingredient for medicaments used in the prevention and treatment of fibrosis. By applying an amount of these HGF medicaments sufficient to inhibit procollagen formation at the site of fibrosis, the symptoms of fibrosis can be treated if not prevented. Preferably these medicaments are administered prior to damage to a tissue near a forming collagen mass characteristic of fibrosis, more preferably prior to the formation of a collagen mass characteristic of fibrosis. Amounts of HGF effective in inhibiting collagen formation are as described above, and include doses between about 0.001 mg and about 50 mg per day per patient, preferably about 0.01 mg and about 10 mg per day per patient, and more preferably about 0.05 mg and about 5 mg per day per patient. HGF protein may be administered to a mammal using a variety of techniques, including direct application to the affected tissue or the skin (transdermal application), systemic injection, nebulized inhalation, and oral ingestion.

HGF for use in these medicaments can be isolated or produced from several sources. A factor to consider however in selecting an HGF source is the simplicity of isolating pharmaceutically acceptable HGF preparations. Thus the number of and complexity of steps necessary to isolate pharmaceutical-grade HGF should always be considered when selecting a production process.

By way of example, HGF may be obtained by extraction and purification from suitable mammalian tissue such as liver, spleen, lung, bone marrow, brain, kidney, placenta and the like, blood cells such as platelets, leukocytes and the like, or the plasma and serum of mammals e.g., rat, cow, horse, and sheep (FEBS Letters, 224, 311-316, 1987; Proc. Natl. Acad. Sci. USA, 86, 5844, 1989).

HGF may also be obtained from primary cell cultures produced from tissues as noted above, or from cell lines known to produce HGF. Cells producing HGF typically secrete the molecule, frequently making its isolation in a semi-pure form possible through centrifugation to isolate the culture supernatant. Cell culture techniques are however notoriously difficult to scale up using present technology, making isolation of large quantities of HGF by this technique impracticable.

HGF also may be obtained by recombinant methods. Briefly, recombinant approaches place a nucleic acid encoding HGF in a suitable vector, the vector then being used to transduce or transfect a suitable cellular host. Suitable hosts may include bacteria, but are preferably eukaryotic cells, as the latter cell types typically possess the cellular machinery to process the recombinant translation product into active HGF. Once the re recombinant host is formed, it may be cultured and HGF harvested as discussed for cell cultures in general, above. (See, e.g. Sambrook et al., Molecular Cloning: A Laboratory Manual, 1989, Cold Spring Harbor Laboratory, New York, USA; Morrison, J. Bact., 132:349-351 (1977); and Clark-Curtiss & Curtiss, Methods in Enzymology, 101:347-362 (Wu et al., eds, 1983); Nature, 342, 440, 1989; Biochem. Biophys. Res. Commun., 163, 967, 1989). For cell culture systems, including recombinant systems, eukaryotic cells are preferred, even more preferably mammalian cells, for the reasons noted above. The host cell however is not specifically limited, and various host cells conventionally used in cell culture and recombinant cell culture methods can be used, including for example bacteria (e.g., Escherichia coli, Bacillus subtilis), yeast, filamentous fungi, and plant or animal cells.

HGF and nucleic acids encoding HGF may also be available commercially, or may be produced commercially, given the structural and/or functional properties of the molecules desired.

B. Fusion Proteins with Increased Half-life

HGF fusion proteins of the present invention are designed to increase the half-life of HGF activity in vivo, without substantially decreasing molar specific activity (activity/mol). In this manner the therapeutic impact of the medicaments of the invention are increased. For example, a region of additional amino acids, particularly charged amino acids, may be added to the N-terminus of the polypeptide to improve stability and persistence during purification from the host cell or subsequent handling and storage. Also, peptide moieties may be added to the HGF protein to facilitate purification. Such regions may be removed prior to final preparation of the polypeptide. The addition of peptide moieties to facilitate handling of polypeptides are familiar and routine techniques in the art.

Moreover, HGF protein of the invention can be combined with parts of the constant domain of immunoglobulins (IgG), resulting in chimeric polypeptides. These fusion proteins both facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).). EP-A-O 464 533 (CA 2045869) discloses fusion proteins comprising various portions of constant region of immunoglobulin molecules together with another human protein or part thereof.

C. Nucleic Acids

Several embodiments of the present invention use gene therapy techniques to deliver the therapeutic effects of the present invention. One aspect of these techniques is possession of a nucleic acid encoding HGF, This nucleic acid, or “HGF gene” can be any gene capable of expressing HGF. Thus, so long as a polypeptide expressed from the gene has substantially the same activity as that of HGF, the HGF gene may have a partial deletion, substitution or insertion of the nucleotide sequence, or may have other nucleotide sequence ligated therewith at the 5′-terminus and/or 3′terminus thereof. Typical examples of such HGF genes include HGF genes as described in Nature, 342, 440 (1989), Japanese Patent KOKAI (Laid-Open) No. 5-111383, Biohem. Biophys. Res. Commun., 163, 967 (1989). These genes may be used in the present invention.

In general, nucleic acid sequences encoding HGF may be isolated from any suitable tissue source, for example suitable mammalian tissues include liver, spleen, lung, bone marrow, brain, kidney, placenta and the like, blood cells such as platelets, leukocytes and the like, or the plasma and serum of mammals e.g., rat, cow, horse, and sheep. Generally, cDNA or genomic libraries are constructed and screened to identify the correct sequence. (For cDNA libraries, see e.g., Gubler & Hoffman, Gene, 25:263-269 (1983); Sambrook et al., 2001, Molecular Cloning: A Laboratory Manual (3^{rd} ed.); Cold Spring Harbor Laboratory, Cold Spring Harbor, N.Y; Ausubel et al. (eds.), 1993, Current Protocols in Molecular Biology, John Wiley & Sons, NY. For genomic libraries, see Benton & Davis, Science, 196:180-182 (1977); Grunstein et al., Proc. Natl. Acad. Sci. USA., 72:3961-3965 (1975); and Gussow, D. and Clackson, T., Nucl. Acids Res., 17:4000 (1989).)

PCR amplification techniques can also be used to identify and isolate nucleic acid sequences encoding HGF, as described in the Examples section below, and more generally in PCR Protocols: A Guide to Methods and Applications (Innis et al., eds, 1990).

Nucleic acids encoding HGF may also be available commercially, or prepared using synthetic techniques well-known to those of skill in the art. See Sambrook, J. et al. Molecular Cloning, A Laboratory Manual, 2d Ed. Cold Spring Harbor Laboratory Press, New York, 13.7-13.9 and Hunkapiller, M. W. (1991) Curr. Op. Gen. Devl. 1:88-92.

### III. Gene Therapy Techniques

In a specific embodiment, nucleic acids comprising sequences encoding HGF, or active variants thereof, are administered to treat, inhibit or prevent fibrosis through gene therapy. The nucleic acids encoding HGF are delivered to the individual suffering fibrosis by either directly applying the nucleic acids to the individual, or by transducing cells ex vivo, the transduced cells then being applied to the suffering individual where they produce HGF, mediating their therapeutic effect. Any of the methods for gene therapy available in the art can be used according to the present invention. Exemplary methods are described below.

In a preferred aspect, the medicaments of the present invention include nucleic acid sequences encoding HGF, more preferably HGF genes capable of expressing HGF. In particular, such nucleic acid sequences have promoters operably linked to the HGF coding region. The promoter may be inducible or constitutive, and, optionally, tissue-specific. In another particular embodiment, the nucleic acid encoding HGF and any other desired sequences are flanked by regions that promote homologous recombination at a desired site in the genome, thus providing for intrachromosomal expression of the HGF encoding nucleic acids (see e.g., Thomas, K. R. and Capecchi, M. R. (1987) Cell 51:503 for a description of homologous recombination cassettes vectors).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

A. DNA Vectors for HGF Expression and Transduction Methods

In a specific embodiment, the nucleic acids encoding HGF are directly administered in vivo, where they express amounts of HGF effective to inhibit collagen formation. Amounts of HGF effective in inhibiting collagen formation are as described above and include doses between about 0.001 mg and about 50 mg per day per patient, preferably about 0.01 mg and about 10 mg per day per patient, and more preferably about 0.05 mg and about 5 mg per day per patient. This can be accomplished by any of numerous methods known in the art, e.g., by constructing an appropriate nucleic acid expression vector and administering it so that the HGF-encoding nucleic acids become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. When the disease is systemic sclerosis or scleroderma, the HGF gene is preferably inserted in a plasmid.

In another embodiment, nucleic acid-ligand complexes can be formed where the ligand comprises a fusogenic viral peotide to disrupt endosomes, allowing the nucleic acid to avoid lysosomal degradation. For example, a preferred embodiment of the present invention uses the hemagglutinating Virus of Japan Envelope (EVJ-E). In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

Liposomes

Liposomes containing the HGF gene of the present invention may be prepared, for example, by suspending a thin layer of purified phospholipids in a solution containing the HGF gene and then treating the suspension in a conventional manner such as ultrasonication. A “Liposome” is a closed vesicle of lipid bilayer encapsulating an aqueous compartment therein. It is known that the lipid bilayer membrane structure is extremely similar to biological membranes. To prepare the liposomes of the present invention, phospholipids are employed. Typical examples of phospholipids are phosphatidylcholines such as lecithin, lysolecithin, etc.; acidic phospholipids such as phosphatidylserine, phosphatidylglycerol, phosphatidylinositol, phosphatidylic acid, etc.; or phospholipids obtained by replacing an acyl group(s) of these acidic phospholipids with lauroyl, myristoyl, oleoyl, etc.; and sphingophospholipids such as phosphatidylethanolamine, sphingomyelin, etc. Neutral lipids such as cholesterol may also be added to these phospholipids. The liposomes may be prepared, in a conventional manner, from naturally occurring materials such as lipids in normal cell membranes.

The liposomes containing the HGF gene be appropriately fused to viruses, etc. to form membrane fusion liposomes. In this case, it is preferred to inactivate viruses, e.g., through ultraviolet irradiation, etc. A particularly preferred example of the membrane fusion liposome is a membrane fusion liposome fused with Sendai virus (hemagglutinating virus of Japan: HVJ). The membrane fusion liposome may be produced by the methods as described in NIKKEI Science, April, 1994, pages 32-38; J. Biol. Chem., 266 (6), 3361-3364 (1991), etc. In more detail, the HVJ-fused liposome (HVJ-liposome) may be prepared, e.g., by mixing purified HVJ inactivated by ultraviolet irradiation, etc. with a liposome suspension containing the HGF gene vector, gently agitating the mixture and then removing unbound HVJ by sucrose density gradient centrifugation. The liposomes may be bound to substances having an affinity to target cells, thereby to enhance an efficiency of gene introduction into the target cells. Examples of substances having an affinity to target cells include ligands such as an antibody, a receptor, etc.

Viral Vectors

Viral vectors may also be used to deliver nucleic acid sequences encoding HGF. For example, a retroviral vector can be used (see Miller et al., 1993, Meth. Enzymol. 217:581-599). Retroviral vectors are typically modified to delete retroviral sequences that are not necessary for packaging of the viral genome and integration into host cell DNA. The nucleic acid sequences encoding HGF are cloned into one or more vectors, facilitating delivery of the gene into a patient. References illustrating the use of retroviral vectors are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses can also be used to deliver nucleic acids encoding HGF. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrate the use of adenovirus vectors to transfer genes to the respiratory epithelia of rhesus monkeys. Other instances of the use of adenoviruses can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

In cases where an adenovirus is used as an expression vector, the HGF coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing HGF infected hosts. (e.g., see Logan & Shenk, 1984, Proc. Natl. Acad. Sci. USA 81:355-359). HGF should be at a level sufficient to inhibit collagen formation associated with fibrosis in the tissue.

Adeno-associated virus (AAV) may also be used to deliver HGF encoding nucleic acids to a mammal suffering from fibrosis (See, e.g., Walsh et al., 1993, Proc. Soc. Exp. Biol. Med. 204:289-300; U.S. Pat. No. 5,436,146).

Expression Control Sequences

Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In order for the nucleic acids encoding HGF to express HGF protein in vivo in amounts sufficient to inhibit collagen formation associated with fibrosis, the nucleic acids must be operably linked to suitable control sequences. Control sequences can allow constitutive expression of HGF, or can direct HGF production to particular tissues, tissue enviromnents, signaling molecules and even temporal control of expression. Such control sequences and their use are well-known to those of skill in the art. Methods include in vitro recombinant DNA techniques and synthetic techniques. See, for example, the techniques described in Sambrook, et al., 1992, Molecular Cloning, A Laboratory Manual, Cold Spring Harbor Laboratory, N.Y. and Ausubel et al., 1989, Current Protocols in Molecular Biology, Greene Publishing Associates & Wiley Interscience, N.Y.

Nucleic acid molecules encoding HGF may be operatively associated with a variety of different promoter/enhancer elements. The promoter/enhancer elements may be selected to optimize for the expression of therapeutic amounts of protein. The expression elements of these vectors may vary in their strength and specificities. Depending on the host/vector system utilized, any one of a number of suitable transcription and translation elements may be used. The promoter may be in the form of the promoter that is naturally associated with the gene of interest, perhaps modified to enhance expression. Alternatively, the DNA may be positioned under the control of a recombinant or heterologous promoter, i.e., a promoter that is not normally associated with that gene. For example, tissue specific promoter/enhancer elements may be used to regulate the expression of the transferred DNA in specific cell types.

Examples of transcriptional control regions that exhibit tissue specificity which have been described and could be used include, but are not limited to, elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:42S-5 1S); insulin gene control region which is active in pancreatic beta cells (Hanahan, 1985, Nature 315:115-122); immunoglobulin gene control region which is active in lymphoid cells (Grosschedl et al., 1984, Cell 38:647-658; Adams et al., 1985, Nature 318:533-538; Alexander et al., 1987, Mol. Cell. Biol. 7:1436-1444): albumin gene control region which is active in liver (Pinkert et al., 1987, Genes and Devel. 1:268-276) α-fetoprotein gene control region which is active in liver (Krumlauf et al., 1985, Mol. Cell. Biol. 5:1639-1648; Hammer et al., 1987, Science 235:53-58); α-1-antitrypsin gene control region which is active in liver (Kelsey et al., 1987, Genes and Devel. 1:161-171); beta-globin gene control region which is active in myeloid cells (Magram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286); and gonadotropic releasing hormone gene control region which is active in the hypothalamus (Mason et al., 1986, Science 234:1372-1378). Promoters isolated from the genome of viruses that grow in mammalian cells, (e.g. vaccinia virus 7.5K, SV40, HSV, adenoviruses MLP, MMTV, LTR and CMV promoters) may be used, as well as promoters produced by recombinant DNA or synthetic techniques.

In some instances, the promoter elements may be constitutive or inducible promoters and can be used under the appropriate conditions to direct high level or regulated expression of the nucleotide sequence of interest. Expression of genes under the control of constitutive promoters does not require the presence of a specific substrate to induce gene expression and will occur under all conditions of cell growth. In contrast, expression of genes controlled by inducible promoters is responsive to the presence or absence of an inducing agent.

Specific initiation signals are also required for sufficient translation of HGF coding sequences. These signals include the ATG initiation codon and adjacent sequences. In cases where the entire coding sequence, including the initiation codon and adjacent sequences are inserted into the appropriate expression vectors, no additional translational control signals may be needed. However, in cases where only a portion of the coding sequence is inserted, exogenous translational control signals, including the ATG initiation codon must be provided. Furthermore, the initiation codon must be in phase with the reading frame of the protein coding sequences to ensure translation of the entire insert. These exogenous translational control signals and initiation codons can be of a variety of origins, both natural and synthetic. The efficiency of expression may be enhanced by the inclusion of transcription attenuation sequences, enhancer elements, etc.

B. Cell-based Systems for HGF Expression

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient. The present invention provides mesenchymal cell preparations that include one or more cells each having a nucleic acid encoding HGF. When introduced to a mammal, these preparations express and secrete HGF in an amount effective to inhibit collagen formation associated with fibrosis. The amount of HGF that must be produced by a given preparation is application and tissue dependent. Methods for making dosage determinations are well known in the art and can be made without undue experimentation. Mesenchymal preparations of the invention include recombinant stem cell and fibroblast cultures.

For cell-based systems, the nucleic acid is introduced into a cell prior to administration in vivo of the resulting recombinant cell. Introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. Care should be taken to ensure that the cell-based systems of the invention have the nucleic acid encoding HGF operably linked to suitable control sequences, as discussed supra.

The resulting recombinant cells can be delivered to a patient by various methods known in the art. Recombinant blood cells (e.g., mesenchymal stem or progenitor cells) are preferably administered intravenously, but depending upon the treatment, may be administered to a mammal using a variety of techniques, including direct application to the affected tissue or the skin (transdermal application), systemic injection, nebulized inhalation, and oral ingestion. The amount of cells envisioned for use depends on the desired effect, patient state, etc., and can be determined by one skilled in the art.

Cells into which a nucleic acid encoding HGF can be introduced for purposes of gene therapy encompass any desired available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in mesenchymal stem or progenitor cells, e.g., as obtained from bone, umbilical cord blood, peripheral blood, fetal tissue, etc, and fibroblasts. Preferably, the cells used in ex vivo techniques as described are autologous to the patient.

In a preferred embodiment, nucleic acid sequences encoding HGF are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid encoding HGF is operably linked to an inducible promoter, such that expression of the nucleic acid is controllable by controlling the presence or absence of the appropriate inducer of transcription. Other embodiments include tissue specific promoters that only produce HGF from an operably linked coding sequence when the construct is positioned in the correct cell type and/or present in the correct tissue environment.

### IV. Methods of Identifying Therapeutically Effective Ranges

The present invention identifies the presence of c-met on the surface of abnormal fibroblasts associated with excessive collagen deposition associated with fibrosis. (See examples 1 and 4, and FIG. 1). The c-met receptor is not present in normal fibroblasts. Kajihara T, et al., Arch Oral Biol 1999;44: 135-47. This finding allows treatment of abnormal (SSc) fibroblasts independent of normal fibroblasts. In order to be therapeutically effective however, the active ingredient of any medicament must present in sufficient quantity to produce the desired effect. Although methods for determining therapeutically effective doses are well-known (including phase I, II and III clinical trials) and can be practiced with only routine experimentation, the following exemplary methods are offered to clarify the invention.

In particularly preferred embodiments, an effective dose range is determined by one skilled in the art using data from routine in vitro and in vivo studies well known to those skilled in the art. For example, in vitro cell culture assays, such as the assays for procollagen production, are described in the examples section, below, and will provide data from which one skilled in the art may readily determine the mean inhibitory concentration (IC) of HGF on procollagen production (such as 50%, IC₅₀; or 90%, IC₉₀). Appropriate doses can then be selected by one skilled in the art using pharmacokinetic data from one or more routine animal models, so that a minimum concentration (Cₘᵢₙ) of HGF is obtained which is equal to or exceeds the determined IC value. Although patient characteristics will impact dosage, others using this sort of approach have reported doses between 0.0001 mg and about 500 mg HGF. U.S. Pat. Nos: 6,248,722; and 5,840,311.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is known as the therapeutic index and can be expressed as the ratio LD₅₀/ED₅₀. Compounds that exhibit large therapeutic indices are preferred. The data obtained from such cell culture assays and animal studies can be used in formulating a range of effective dosages for use in humans or other mammals, particularly domesticated animals including farm animals. The dosage lies preferably within a range of concentrations that include the ED₅₀ with little or no toxicity. The dose may vary within this range depending upon the dosage form employed and the route of administration utilized. For any medicament administered as part of the methods and compositions of this invention (i.e., for any therapeutic reagent), the therapeutically effective dose can be estimated initially from cell culture assays. A dose may be formulated in animal models to achieve a concentration range that includes the IC₅₀ as determined in cell culture. Such information can be used to more accurately determine useful doses in humans and other mammals. Levels in plasma may be measured, for example, by high performance liquid chromatography (HPLC) or by any biological or immunological assay capable of measuring levels of a therapeutic reagent.

The methods and compositions of the invention may be used to administer HGF, or derivatives thereof, intermittently, periodically or continuously. For example, hydrogel or other slow or control release excipients or systems may be used to administer therapeutic reagents in a single administration such as a direct application to the skin for transdermal administration, direct application to an affected tissue, systemic injection, nebulized inhalation, and oral ingestion. Viral vectors and other gene therapy methodologies are particularly suited to single or single repeat dosing.

The medicaments of the present invention may also be delivered in a plurality of intermittent administrations, including periodic administrations. For example, in certain embodiments HGF, or a nucleic acid form including the coding sequence for HGF, can be administrated annually (i.e., once a year), semiannually (i.e., once every six months), once a trimester (i.e., once every 4 months), once a quarter (i.e., once every 3 months), bimonthly (i.e., once every two months) or once a month. HGF or transient gene therapy medicaments may also be administered more frequently using the methods and compositions of the invention, such as once a week, once a day, twice a day (e.g., every 12 hours), every six hours, every four hours, every two hours, or every hour. In some circumstances, multiple transductions using the gene therapy techniques described above are advisable, as a surprising result of the present invention is the inhibition of SSc fibroblast procollagen production by high HGF concentrations. Multiple transductions may provide a means to produce the maximal number of HGF-producing recombinant cells, or may increase the copy number of active HGF genes in the target cells.

### V. Method for Early Diagnosis of Fibrosis

The present invention also provides methods for the early diagnosis of fibrosis. As described above, fibrosis is characterized by excessive buildup of fibrous tissue, particularly collagen. In a number of tissues this buildup of connective tissue can result in damage to surrounding tissue, which either slowly heals or never heals completely. By allowing for the early detection of fibrosis, the methods of the present invention provide a means to treat the disease before debilitating tissue damage can occur. These detection methods typically involve identifying the expression of c-met mRNA or protein in the suspect fibroblasts.

As described in examples 1 and 4, abnormal fibroblasts express and display the HGF receptor c-met at a very early stage of the disease process, necessarily while the disease is still asymptomatic. Through early screening and treatment with the medicaments of the present invention, the formation of collagen masses characteristic of fibrosis and associated collateral tissue damage may be ablated.

One exemplary embodiment for the early screening of susceptible individuals includes the steps of extracting total RNA from fibroblasts taken by biopsy from the suspect tissue. RT-PCR (Sambrook et al., 2001, Molecular Cloning: A Laboratory Manual (3^{rd} ed.); Cold Spring Harbor Laboratory, Cold Spring Harbor, N.Y.; Ausubel et al. (eds.), 1993, Current Protocols in Molecular Biology, John Wiley & Sons, NY.) is performed and the products evaluated. Methods for taking tissue biopsies and peparing them for RT-PCR are well-known to those of skill in the art. The presence of c-met, as for example determined by the presence of a characteristic molecular weight band upon agarose gel electrophoresis, is indicative of abnormal fibroblasts that can lead to fibrosis. Once identified, treatment with one or more of the medicaments provided by the present invention may be used to prevent the onset of fibrosis. Results can be confirmed by sequencing the nucleic acids produced by the RT-PCR amplification, using techniques well known in the art.

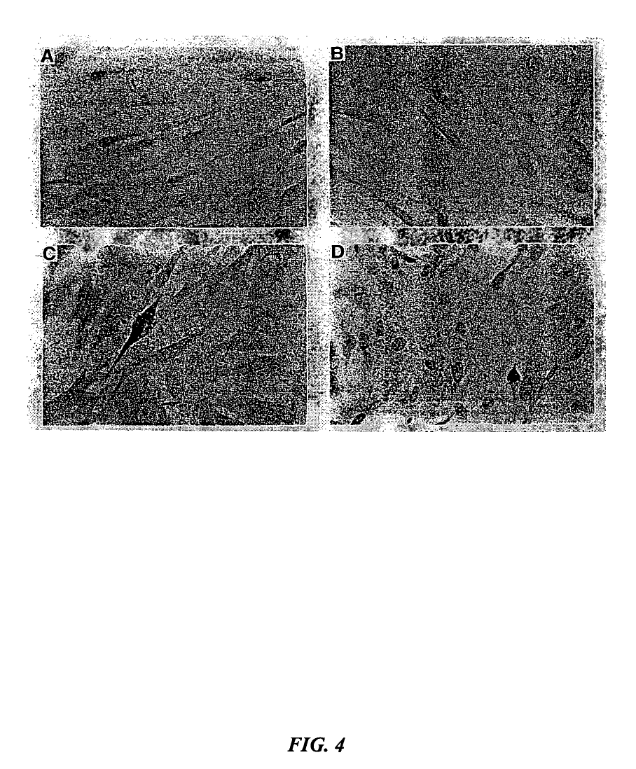

A second exemplary method for early diagnosis of fibrosis is presented in example 4. This approach identifies the presence of c-met protein on the cell surface of suspect fibroblasts using immunostaining techniques. Fibroblasts are first isolated from the suspect tissue and cultured using methods well-known in the art. The cultured fibroblasts are then immunostained by, for example, the method described in the examples section, below. Representative results using this technique are shown in FIG. 4. Panels A and B show the expression of c-met in fibroblasts derived from 2 normal individuals, and panels C and D showed that in fibroblasts from 2 patients with SSc. Both SSc fibroblast lines expressed c-met, but the expression of c-met was not detected at all in normal fibroblasts. Preferably, any dosing using the medicaments of the present invention will be administered prior to damage to a tissue near a forming collagen mass characteristic of fibrosis, more preferably administered prior to the formation of a collagen mass characteristic of fibrosis

### VI. Therapeutic Use of Medicaments in Treatment of Fibrosis in Mammals

The medicaments and methods of the present invention are useful in preventing or treating fibrosis. The number of disease states in which fibrosis can occur and are treatable using the present medicaments and methods is long and includes systemic sclerosis, scleroderma, dermatosclerosis, sclerosis corii, sclerosis cutanea, localized scleroderma, morphea, scleroderma circumscriptum, sclerodermatitis, hidebound disease and skin bound disease.

Pharmaceutically Acceptable Excipients

The medicaments are administered to a mammal, preferably a human, and contain a pharmaceutically-acceptable excipient, or carrier. Suitable excipients and their formulations are described in Remington's Pharmaceutical Sciences, 16th ed., 1980, Mack Publishing Co., edited by Oslo et al. Typically, an appropriate amount of a pharmaceutically-acceptable salt is used in the formulation to render the formulation isotonic. Examples of the pharmaceutically-acceptable excipients include liquids such as saline, Ringer's solution and dextrose solution. The pH of the solution is preferably from about 5 to about 8, and more preferably from about 7 to about 7.5. The formulation may also comprise a lyophilized powder or other optional excipients suitable to the present invention including sustained release preparations such as semipermeable matrices of solid hydrophobic polymers, which matrices are in the form of shaped articles, e.g., films, liposomes or microparticles. It will be apparent to those persons skilled in the art that certain excipients may be more preferable depending upon, for instance, the route of administration the concentration of HGF being administered, or whether the treatment uses a medicament having HGF protein, a nucleic acid encoding HGF or a cell capable of secreting HGF as the active ingredient.

Methods of Administering Medicaments

The medicaments described herein may be administered a the mammal by systemic injection, direct application of the medicament to the organ by infusion, injection, or bathing, transdermal administration by applying the medicament directly to the skin, nebulized inhalation, oral ingestion, or by other methods such as systemic infusion that ensure delivery of the active ingredient to the site of fibrosis, or potential fibrosis injury, in an effective form. Commercially available nebulizers for liquid formulations, including jet nebulizers and ultrasonic nebulizers may be useful for administration. Liquid formulations may be directly nebulized and lyophilized power nebulized after reconstitution. Alternatively, the medicaments may be aerosolized using a metered dose inhaler, or inhaled as a lyophilized and milled powder. In addition, a liquid medicament may be directly instilled in the nasotracheal or endotracheal tubes in intubated patients.

Effective dosages and schedules for administering the medicament may be determined empirically, and making such determinations is within the skill in the art, as described above. Those skilled in the art will understand that the dosage of medicament that must be administered will vary depending on, for example, the mammal receiving the medicament, the route of administration, the particular type of medicament used and other drugs being administered to the mammal. As previously noted, the medicaments of the present invention may be administered in a single dose, or as multiple doses over time. Preferably the medicament is administered prior to damage to a tissue near a forming collagen mass characteristic of fibrosis, more preferably prior to the formation of a collagen mass characteristic of fibrosis.

Optional Additives

The medicaments of the present invention may optionally include other pharmacologic agents used to treat the conditions listed above, such as UTP, amiloride, DNase, antibiotics, bronchodilators, anti-inflammatory agents, and mucolytics (e.g. n-acetyl-cysteine). It may also be useful to include in the medicament therapeutic human proteins such as protease inhibitors, gamma-interferon, enkephalinase, lung surfactant, and colony stimulating factors. In addition to including other therapeutic agents in the medicament itself, the medicaments of the present invention may also be administered sequentially or concurrently with the one or more other pharmacologic agents. The amounts of medicament and pharmacologic agent depend, for example, on what type of pharmacologic agent(s) is are used, the disease being treated, and the scheduling and routes of administration.

Following administration of a medicament of the invention, the mammal's physiological condition can be monitored in various ways well known to the skilled practitioner.

### VII. Kits

In another embodiment ofthe invention, there are provided articles of manufacture and kits containing materials useful for treating the pathological conditions described herein. The article of manufacture comprises a container of a medicament as described herein with a label. Suitable containers include, for example, bottles, vials, and test tubes. The containers may be formed from a variety of materials such as glass or plastic. The container holds a composition having an active agent which is effective for treating, for example, systemic sclerosis, scleroderma, dermatosclerosis, sclerosis corii, sclerosis cutanea, localized scleroderma, morphea, scleroderma circumscriptum, sclerodermatitis, hidebound disease or skin bound disease. The active agent in the composition is HGF, or an agent such as a vector or a cell preparation capable of allowing production of HGF in vivo. The label on the container indicates that the composition is used for treating fibrosis and may also indicate directions for administration and monitoring techniques, such as those described above.

The kit of the invention comprises the container described above and a second container comprising a pharmaceutically-acceptable diluent. It may further include other materials desirable from a commercial and user standpoint, including other buffers, diluents, filters, needles, syringes, and package inserts with instructions for use.

### EXAMPLES

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### General Methods

The following methods are general to all examples that follow.

Patients

Skin biopsies were performed for 6 patients with systemic sclerosis whose diagnoses met the classification for SSc of the American College of Rheumatology (formerly, the American Rheumatism Association, See, e.g., Subcommittee for Scleroderma Criteria of the American Rheumatism Association Diagnostic and Therapeutic Criteria Committee: Preliminary criteria for the classification of systemic sclerosis (scleroderma). Arthritis Rheum 1980;23:581-90). All were classified as diffuse cutaneous type according to the classification of LeRoy and colleague (LeRoy E C, et al., J Rheumatol 1988;15:202-5). Informed consent was obtained from all patients enrolled. The characteristics of the patients enrolled in the study are shown in Table 1.

TABLE 1
Characteristics of SSc patients and controls

| | | | | |
|---|---|---|---|---|
| | | Duration of disease | | |
| | Age | prior to biopsy | | |
| Sex | (yrs) | (months) | ANA | Topo I |

SSc patients

| | | | | | |
|---|---|---|---|---|---|
| 1 | F | 52 | 6 | 640 sp | + |
| 2 | F | 20 | 7 | 2560 sp | + |
| 3 | F | 58 | 16 | 640 sp. nu | − |
| 4 | F | 63 | 10 | 640 sp. nu | + |
| 5 | F | 47 | 6 | 5120 sp. nu | + |
| 6 | M | 45 | 14 | 320 nu | − |

Controls

| | | |
|---|---|---|
| 1 | F | 38 |
| 2 | F | 46 |
| 3 | F | 52 |
| 4 | F | 40 |

ANA: antinuclear antibody determined by immunofluorescence staining; sp: speckled pattern; nu: nucleolar pattern. Topo I: antitopoisomerase I antibody determined by immunodiffusion.

Cell Culture

Skin explants were carried out following a method as described previously (Kawaguchi Y., Clin Exp Immunol 1994;97:445-50). The explants were derived from skin biopsies from affected and unaffected (confirmed by pathological exam) skin from SSc patients and from normal healthy donors. The minced skin was placed in plastic dishes (Corning Glass Works, Corning, N.Y.). After attachment, culture medium consisting of Dulbecco's modified essential medium (DMEM, Flow Laboratories, McLean, Va.) with 10% fetal bovine serum (FBS, Filtron, Brisbane, Australia), 10 units/ml of penicillin, and 10 μg/ml streptomycin (Gibco, Grand Island, N.Y.) was added to the dishes. Explant cultures were incubated at 37° C. in a 5% CO₂ incubator, and then subcultures were established by trypsinization (0.25%, Sigma, St. Louis, Mo.) of the primary cultures. The cells in the 3rd through 5th passages were used in this study.

Plasmid Construction and Stable Transfection

We obtained a full-length 2.4 kb human IL-1α (IL-1α) cDNA from the American Type Culture Collection (Bethesda, Md.). The cDNA insert was excised by BamHI and subcloned into the pcDNA3 vector (Invitrogen, San Diego, Calif.) to create the IL-1α sense-encoding construct (pcDNA3-IL-1α). This construct confers resistance to neomycin (G418), and expression of the insert is driven by the human cytomegalovirus promoter. Stable transfection was performed as described by Felgner and colleague (Felgner PL, et al., Proc Natl Acad Sci USA 1987;84:7413-7). Briefly, 2 μg of DNA (twice cesium chloride-banded) and 8 μl of LipofectAMINE (Gibco) were added to 1 ml of Opti-MEM (Gibco), and this mixture was added to fibroblasts (5×10⁴) on 35 mm culture dishes. Seventy-two h after transfection, medium was replaced and cells were trypsinized and transferred to ten 100 mm dishes with 10 ml of complete medium supplemented with 450 μg/ml of G418 (Gibco). Continuous G418 selection for approximately 4 weeks resulted in generation of drug-resistant colonies. Individual colonies were harvested using cloning rings and expanded for further analysis.

Determination of Hepatocyte Growth Factor and Procollagen Type I C-peptide

Fibroblasts (2×10⁴ cells/well) were cultured in 24-well culture plates (Linbro, Flow Laboratories) with DMEM plus 10% FBS. After confluency had been reached, culture medium was discarded and each well was washed with PBS twice, and then serum-free medium (QBSF-51, Sigma) with various concentrations of recombinant IL-1α (Genzyme, Cambridge, Mass.) or various concentrations of recombinant HGF (R & D Systems, Minneapolis, Minn.) was added to 24-well culture plates. The supernatants of each well were collected and stored at −80° C. until use. HGF concentrations in culture supernatants were measured by an enzyme-linked immunosorbent assay (ELISA) system developed by Otsuka Pharmaceutical Co. (Tokushima, Japan). The concentrations of procollagen type I C-peptide in culture supernatants were measured by an ELISA system (Takara Shuzo, Otsu, Japan), the results of which correlates well with the production of collagen type I.

Immunocytochemical Staining

Fibroblasts (5×10⁴ cells/well) were grown for 48 h in serum-free medium on 4-chamber slides (Lab-tek, Nunc Inc., Naperville, Ill.). Fibroblasts were washed twice with cold-PBS, and fixed with 2% PFA in PBS. The primary antibody used in these experiments was a polyclonal goat anti-c-met antibody (Santa Cruz, Santa Cruz, Calif.). Cells were incubated with the primary antibody (10 μg/ml) or preimmune goat IgG (10 μg/ml, Immunovision, Springdale, Ariz.) as a control for 1 h at 4° C. The primary antibody was detected by incubation with biotinylated anti-goat IgG (H+L) antibody (Pierce, Rockford, Ill.) for 30 min at room temperature, followed by incubation with avidin:biotinylated enzyme complex (ABC, Pierce) and development with 3,3′diaminobenzidine tetrahydrochloride (DAB) peroxidase substrate (SIGMA FAST™, Sigma) for 10 min. The chamber slides were dried and examined by light microscopy.

RNA Isolation and Reverse Transcriptase (RT)-PCR

Total RNA was extracted from fibroblasts using the Trizol™ reagents (Gibco Laboratories, Grand Island, N.Y.). RT-PCR was performed using an RNA PCR kit (Perkin-Elmer Cetus, Norwalk, Conn.). Briefly, 1 μg of total RNA from each sample was reverse transcribed to cDNA in the reaction buffer including 10 mM Tris-HCl, pH 8.3, 50 mM KCl, 5 mM MgCl₂, 1 mM of each dNTP and 500 ng OligodT; the RT reaction was performed at 42° C. for 30 min in a volume of 20 μl. Two microliters of each RT solution were then used for PCR in a volume of 50 μl containing MgCl₂ (final 1.5 mM), a set of primers (0.2 mM each), and 2.5 U of AmpliTaq polymerase. For analysis of transcription of HGF and c-met, 35 cycles of PCR were performed with denaturation at 94° C. for 1 min, annealing at 56° C. for 1 min, and extension at 72° C. for 1.5 min. We used three sets of primers, for:

HGF
sense: 5′-CTGATCCAAACATCCGAGTTGG-3′,
antisense: 5′-AGGTGTGGTATCACCTTCACAACG-3′,
product size: 313 bp;
c-met
sense: 5′-TCCTCGTGCTCCTGTTTACC-3′,
antisense: 5′-TCTTTCGTTTCCTTTAGCCTTC-3′,
product size: 638 bp;
and,
β-actin (used as a control)
sense: AAGAGAGGCATCCTCACCCT-3′,
antisense: 5′-TACATGGCTGGGGTGTTGAA-3′,
product size: 218 bp

A 10 μl aliquot of each PCR sample was resolved by electrophoresis in 2% agarose gels with loading DNA marker (100 bp ladder, New England Biolabs, Beverly, Mass.). For confirming the sequences of the PCR products, the direct sequencing using ABI PRISM 7700 Sequence Detection System (Applied Biosystems, CA) was performed.

Statistical Analysis

Results are expressed as means ±SD. Statistical comparisons were performed using the Mann-Whitney U test. P values <0.05 were considered significant for all tests.

### RESULTS

### Example 1

### Expression of HGF and c-met mRNA in Cultured Fibroblasts Derived From SSc Patients and Normal Healthy Individuals

To determine whether cultured fibroblasts expressed the mRNA of HGF and c-met constitutively, RT-PCR was performed. Total RNA was extracted from 6 SSc patients and three normal donors. As shown in FIG. 1, both SSc and normal fibroblasts expressed HGF mRNA constitutively. However, the expression of c-met mRNA was constitutive only in SSc fibroblasts, and was undetectable in normal fibroblasts (FIG. 1). The direct sequencings for the PCR products of HGF and c-met indicated the amplification of the consensus sequences of those genes (data not shown). In FIG. 1, total RNA was extracted from cultured fibroblasts from 6 patients with SSc and 3 healthy individuals. The extracted RNA was analyzed by RT-PCR using primers specific for human HGF, c-met, and β actin (internal control).

### Example 2

### HGF Production of Cultured Fibroblasts Derived From SSc Patients and Normal Healthy Individuals

Fibroblasts (3×10⁴) were cultured in 24-well culture plates with DMEM plus 10% FBS. After confluency had been reached, medium was discarded and serum-free medium (QBSF-5 1) was added at Time 0. A time-course study was performed without stimulation as shown in FIG. 2. HGF production of fibroblasts from 2 SSc patients and 2 normal healthy individuals was measured using ELISA. In both groups, the production of HGF increased steadily up to 120 h. Measured amounts of HGF production by SSc fibroblasts were larger than those for healthy controls at every stage throughout the culture period (FIG. 2). Especially, the ratio of increasing HGF production appoximately reached to a plateau at 48 h of incubation, as illustrated by FIG. 2. On the basis on these results, the experiment of HGF production has been performed in a 48 h-incubation of cultured fibroblasts. Total HGF production of the 6 SSc and 3 normal fibroblasts after 48 h culture period is shown in Table 2; there was a significant difference in spontaneous HGF production between SSc and normal fibroblasts (p<0.05, Mann-Whitney U test). Fibroblasts of FIG. 2 were cultured in serum-free medium (QBSF-51) for indicated lengths of time. Cells were obtained from 2 patients with SSc (white symbols) and 2 normal individuals (black symbols).

### Example 3

### Effect of IL-1α on HGF Production in SSc Fibroblasts

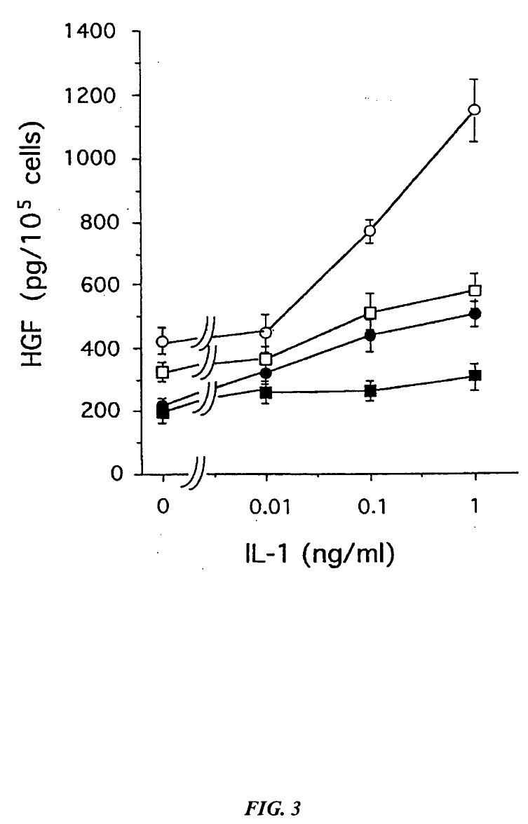

We have reported that IL-1α played a critical role in the fibrotic phenotype of SSc fibroblasts (Kawaguchi Y, et al., J Clin Invest 1999;103:1253-60). To investigate the effect of IL-1α on HGF production in SSc fibroblasts, we measured HGF concentrations in the supernatants of cultured SSc fibroblasts with various concentrations of recombinant IL-1α for 48 h. Representative results are shown in FIG. 3 for 2 lines of SSc fibroblasts and 2 lines of normal controls. To produce this figure, fibroblasts derived from 2 patients with SSc (white symbols) and two normal individuals (black symbols) were cultured in serum-free medium (QBSF-51) with or without recombinant IL-1α. Results indicate the mean ±standard deviation.

A summary of results for fibroblasts from 6 patients with SSc and 4 normal individuals is shown in Table 2, below. HGF production by SSc and normal fibroblasts was significantly increased by the addition of IL-1α.

TABLE 2
HGF production in SSc and normal fibroblasts. Fibroblasts were
cultured in serum-free media with or without IL-1α for 48 h. Values
are the mean ± SD. P values represent comparisons between groups
with and without IL-1α.

| | HGF Production (pg/10⁵ cells) | | |
|---|---|---|---|
| | No stimulation | IL-1a, 1 ng/ml | P |
| SSc fibroblasts | 306 ± 65* | 770 ± 350 | <0.05 |
| n = 6 | | | |
| Normal fibroblasts | 198 ± 20 | 414 ± 101 | <0.05 |
| n = 4 | | | |

*p < 0.05 for HGF production in SSc fibroblasts vs controls. Mann-Whitney U test.

### Example 4

### Immunocytochemistry for c-met Protein in SSc and Normal Fibroblasts

We performed immunostaining for c-met in cultured skin fibroblasts derived from 6 SSc patients and 3 normal controls. Immunostaining was heterogeneusly positive in all lines of SSc fibroblasts. In contrast, normal fibroblasts exhibited no immunostaining of c-met. Representative results are shown in FIG. 4. Panels A and B showed the expression of c-met in fibroblasts derived from 2 normal individuals, and panels C and D showed that in fibroblasts from 2 patients with SSc. Both SSc fibroblast lines expressed c-met, but the expression of c-met was not detected at all in normal fibroblasts.

### Example 5

### Expression of c-met mRNA by IL-1α-transfected Normal Fibroblasts

Five clones of IL-1α-transfected normal fibroblasts were prepared using the standard procedure described in Materials and Methods. These clones exhibited high levels of expression of IL-1α protein constitutively (data not shown). To examine the effect of endogenous IL-1α on c-met expression, we performed RT-PCR for c-met mRNA using total RNA derived from IL-1α-transfected normal fibroblasts. The specific band appeared in 5 clones of IL-1α-transfected normal fibroblasts.

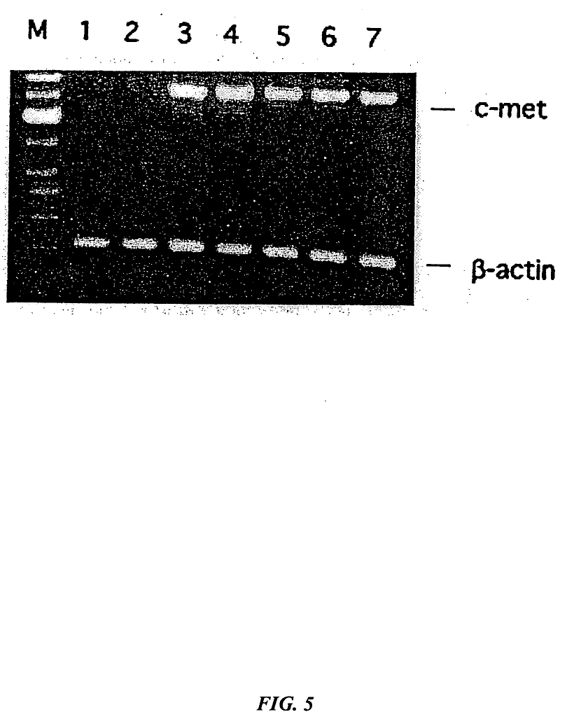

Representative results are shown in FIG. 5. Briefly, expression vectors (pcDNA3) with or without IL-1α cDNA were transfected into normal fibroblasts by lipofection. Total RNA was extracted from fibroblasts and mRNA expression of c-met was estimated by RT-PCR. PCR products were electrophoresed through 2% agarose gels and visualized using ethidium bromide. Lane 1: normal fibroblasts; Lane 2: normal fibroblasts stably transfected with pcDNA3; Lanes 3-7: normal fibroblast lines stably transfected with pcDNA3-IL-1α.

### Example 6

### Effect of HGF on Procollagen Type I Production by Fibroblasts

To examine the function of HGF in fibrotic lesions, we measured procollagen production in cultured fibroblasts derived from 5 patients with SSc and 2 normal controls with various concentrations of HGF. As shown in FIG. 6, procollagen production was significantly and dose-dependently reduced in SSc fibroblasts by the addition of HGF, and this phenomenon was specific to SSc fibroblasts.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

1. A method of treating or preventing fibrosis comprising administering a medicament to a mammal, the medicament comprising:
a) HGF in an amount effective to inhibit collagen formation; and,
b) a pharmaceutically acceptable excipient.

2. The method of claim 1, wherein the amount effective to inhibit collagen formation is between about 0.001 mg about 50 mg per day per patient.

3. The method of claim 2, wherein the amount effective to inhibit collagen formation is between about 0.01 mg and about 10 mg per day per patient.

4. The method of claim 3, wherein the amount effective to inhibit collagen formation is between about 0.05 mg and about 5 mg per day per patient.

5. The method of claim 1, wherein the medicament further comprises an anti-inflammatory agent.

6. The method of claim 1, wherein administering the medicament comprises a technique selected from the group consisting of direct application, systemic injection, nebulized inhalation, and oral ingestion.

7. The method of claim 1, wherein fibrosis is associated with a disease selected from the group consisting of systemic sclerosis, scleroderma, dermatosclerosis, sclerosis corii, sclerosis cutanea, localized scleroderma, morphea, scleroderma circumscriptum, sclerodermatitis, hidebound disease and skin bound disease.

8. The method of claim 1, wherein administering the medicament comprises introducing the medicament through multiple doses over a period of time.

9. The method of claim 1, wherein HGF is modified to increase its half-life after administering the medicament of a mammal.

10. The method of claim 9, wherein HGF is a part of a fusion protein.

11. The method of claim 1, wherein the mammal is a human.

12. The method of claim 1, wherein administering the medicament occurs prior to the formation of a collagen mass characteristic of fibrosis.

13. The method of claim 1, wherein administering the medicament occurs prior to damage to a tissue near a forming collagen mass characteristic of fibrosis.

14. A medicament comprising:
a) a vector including a nucleic acid comprising a nucleotide sequence encoding HGF, wherein introducing the medicament to a subject results in expression and secretion of HGF protein in an amount effective to inhibit collagen formation; and,
b) a pharmaceutically acceptable excipient.

15. The medicament of claim 14, wherein the amount effective to inhibit collagen formation is between about 0.001 mg and about 50 mg per day per patient.

16. The medicament of claim 15, wherein the amount effective to inhibit collagen formation is between about 0.01 mg and about 10 mg per day per patient.

17. The medicament of claim 16, wherein the amount effective to inhibit collagen formation is between about 0.05 mg and about 5 mg per day per patient.

18. The medicament of claim 14, wherein the medicament further comprises an anti-inflammatory agent.

19. The medicament of claim 14, wherein the vector is an EJV virus.

20. A method for treating or preventing fibrosis comprising administering the medicament of claim 14 to a mammal.

21. The method of claim 20, wherein administering the medicament comprises a technique selected from the group consisting of direct application, systemic injection, nebulized inhalation, and oral ingestion.

22. The method of claim 20, wherein fibrosis is associated with a disease selected from the group consisting of systemic sclerosis, scleroderma, dermatosclerosis, sclerosis corii, sclerosis cutanea, localized scleroderma, morphea, scleroderma circumscriptum, sclerodermatitis, hidebound disease and skin bound disease.

23. The method of claim 20, wherein administering the medicament comprises introducing the medicament through multiple doses over a period of time.

24. The method of claim 20, wherein the mammal is a human.

25. The method of claim 20, wherein the medicament is administered prior to the formation of a collagen mass characteristic of fibrosis.

26. The medicament of method of claim 20, wherein the medicament is administered prior to damage to a tissue near a forming collagen mass characteristic of fibrosis.

27. A medicament comprising:
a) a mesenchymal preparation comprising one or more cells each having a nucleic acid comprising a nucleotide sequence encoding HGF, and,
b) a pharmaceutically acceptable excipient.
wherein introducing the medicament to a mammal results in expression and secretion of HGF in an amount effective to inhibit collagen formation.

28. The medicament of claim 27, wherein the amount effective to inhibit collagen formation is between about 0.001 mg and about 50 mg per day per patient.

29. The medicament of claim 28, wherein the amount effective to inhibit collagen formation is between about 0.01 mg and about 10 mg per day per patient.

30. The medicament of claim 29, wherein the amount effective to inhibit collagen formation is between about 0.05 mg and about 5 mg per day per patient.

31. The medicament of claim 27, wherein the mesenchymal preparation comprises stem cells.

32. The medicament of claim 27, wherein the mesenchymal preparation comprises fibroblasts.

33. The medicament of claim 27, wherein the medicament further comprises an anti-inflammatory agent.

34. A method for treating or preventing fibrosis comprising administering the medicament of claim 27 to a mammal.

35. The method of claim 34, wherein administering the medicament comprises introducing the medicament through multiple doses over a period of time.

36. The method of claim 34, wherein the mammal is a human.

37. The method of claim 34, wherein the medicament is administered prior to the formation of a collagen mass characteristic of fibrosis.

38. The method of claim 34, wherein the medicament is administered prior to damage to a tissue near a forming collagen mass characteristic of fibrosis.

39. The method of claim 34, wherein administering the medicament comprises contacting directly with the medicament a tissue having fibrosis.

40. A method for early diagnosis of fibrosis comprising:
detecting the expression of HGF receptor in fibroblasts isolated from a mammal.

41. The method of claim 40, wherein detecting comprises not observing expression of HGF receptor indicating that the mammal does not have fibrosis.

42. The method of claim 40, wherein detecting comprises observing expression of HGF receptor indicating that the mammal is susceptible to fibrosis.

43. The method of claim 40, wherein the detecting step comprises PCR detection of HGF receptor transcript from total RNA isolated from fibroblasts taken from a mammal.

44. The method of claim 40, wherein the HGF receptor is c-met.

45. An article of manufacture comprising:
a) a medicament comprising HGF; and,
b) instructions as to administration of the medicament in a manner and amount sufficient to inhibit formation of a collagen mass characteristic of fibrosis.

46. The article of claim 45, further comprising an inhaler.

47. An article of manufacture comprising:
a) a medicament comprising a vector including a nucleic acid comprising a nucleotide sequence encoding HGF; and,
b) instructions as to administration of the medicament in a manner and amount sufficient to inhibit formation of a collagen mass characteristic of fibrosis.

48. An article of manufacture comprising:
a) a medicament comprising a mesenchymal preparation comprising one or more cells each having a nucleic acid comprising a nucleotide sequence encoding HGF, and,
b) instructions as to culturing and administering the medicament in a manner and amount sufficient to inhibit collagen formation.

49. A method for treatment of a disease selected from systemic sclerosis, scleroderma, dermatosclerosis, sclerosis corii, sclerosis cutanea, localized scleroderma, morphea, scleroderma circumscriptum, sclerodermatitis, hidebound disease or skin bound disease, administering HGF (hepatocyte growth factor) gene.

50. The method defined in claim 49, wherein the disease is systemic sclerosis or scleroderma.

51. The method defined in claim 49 or 50, wherein the disease is systemic sclerosis.

52. The method defined in claim 49 or 50, wherein the disease is scleroderma.

53. The method defined in any claim 49 to 52, wherein the HGF gene is inserted in expression vector.

54. The method defined in claim 53, wherein the expression vector is selected from plasmid, adenovirus vector or EVJ-E (Hemagglutinating Virus of Japan Envelope) vector.

55. The method defined in any claim 49 to 54, wherein the disease is systemic sclerosis or scleroderma and the HGF gene is inserted in plasmid.

56. The method defined in any claim 49 to 55, administering HGF gene transdermally.
